(19) United States
(12) Patent Application Publication
(10) Pub. No.: US 2023/0117747 A1
(43) Pub. Date: Apr. 20, 2023

(54) DEVICES, SYSTEMS, AND METHODS FOR TREATING SEPSIS AND/OR VIRAL INFECTION

(71) Applicant: ADVANCED COOLING THERAPY, INC., Chicago, IL (US)

(72) Inventor: Erik KULSTAD, Chicago, IL (US)

(21) Appl. No.: 17/906,932

(22) PCT Filed: Mar. 26, 2021
(86) PCT No.: PCT/US2021/024353

§ 371 Date: Sep. 21, 2022

Related U.S. Application Data
(60) Provisional application No. 63/000,785, filed on Mar. 27, 2020.

(51) Int. Cl. A61F 7/12

(52) U.S. Cl. CPC A61F 7/12; A61F 2007/0054

(57) ABSTRACT

Methods, devices, and systems for temperature management of a patient having a viral infection or suffering from sepsis are disclosed. Methods, devices, and systems for raising a regional temperature of the patient to and/or actively maintaining a regional temperature of the patient at a target temperature are disclosed. The use of esophageal heat transfer devices to treat patients having a viral infection or suffering from sepsis is disclosed.

### CROSS REFERENCE TO RELATED APPLICATIONS

This application claims priority to U.S. Provisional Patent Application No. 63/000,785, filed on Mar. 27, 2020, the entire contents of which are herein incorporated by reference.

### FIELD OF THE INVENTION

The present application relates to temperature management in patients with sepsis and/or viral infection, including patients infected with SARS-CoV-2, which causes coronavirus disease 2019 (COVID-19). The present application also relates to patient temperature management devices and systems, particularly esophageal temperature management devices and systems.

### BACKGROUND OF THE INVENTION

In humans, thermoregulatory processes maintain body temperature within narrow limits, usually 36.5-37.5° C. Targeted temperature management (“TTM”) is an important component in the care of a patient suffering from severe brain trauma or from ischemia, such as that caused by stroke, heart attack, or cardiac arrest. The medical outcome for such patient suffering is improved if the patient is cooled below normal body temperature (37° C.). Furthermore, it is also accepted that for such patients, it is important to prevent hyperthermia (fever) even if it is decided not to induce hypothermia.

SARS-CoV-2 is the virus responsible for coronavirus disease 2019 (COVID-19) COVID-19 is spreading rapidly across the globe, with no proven effective therapy. Fever is seen in most cases of COVID-19, at least at the initial stages of illness. Although fever is typically treated (with antipyretics or directly with ice or other mechanical means), increasing data suggest that fever is a protective adaptive response that facilitates recovery from infectious illness.

Despite advances in the field, there continues to be a significant need for treatments for viral diseases, particularly viral diseases caused by infection with SARS-CoV-2. Heretofore, there is a paucity of data on temperature management for COVID-19. Indeed, it has recently been suggested by others that maximum body temperature during the course of COVID-19 is a predictor of worse outcomes.

Sepsis affects approximately 1.7 million adults in the U.S. each year and causes over 250 000 deaths annually. Medicare data show a total cost of sepsis care of $62 billion in 2019. Globally, sepsis affects an estimated 49 million people and causes 11 million deaths a year. Even though sepsis is a leading cause of death and among the most expensive conditions treated in hospitals, effective treatments are limited, and mortality of sepsis remains high. Although only half of patients with sepsis have fever, fever is associated with improved survival. The use of external warming techniques, such as forced air warming devices, has been explored in severe septic patients. However, the safety and efficacy of internally placed warming devices cannot be predicted based on external warming, particularly in patients with respiratory failure or at risk of respiratory failure due to viral infection and/or sepsis.

### BRIEF SUMMARY OF THE INVENTION

The present technology provides devices, methods, and systems for rapidly and efficiently managing a regional temperature of a subject with a viral infection and/or suffering from sepsis, while at the same time maintaining access to important anatomical structures. In certain embodiments, the region comprises one or more thoracic structures, such as lungs, bronchi, pulmonary vasculature, and/or heart. In certain embodiments, thoracic temperature is managed using an esophageal heat transfer device.

At least one aspect of the present technology provides one or more methods to treat a patient infected with a virus.

At least one aspect of the present technology provides one or more systems to treat a patient infected with a virus.

At least one aspect of the present technology provides one or more methods to treat a patient suffering from sepsis, including bacterial sepsis.

At least one aspect of the present technology provides one or more systems to treat a patient suffering from sepsis, including bacterial sepsis.

At least one aspect of the present technology provides one or more methods to reduce viral load of a patient infected with a virus.

At least one aspect of the present technology provides one or more systems to reduce viral load of a patient infected with a virus.

At least one aspect of the present technology provides one or more methods to reduce or prevent sepsis-induced immunoparalysis in a septic patient.

At least one aspect of the present technology provides one or more systems to reduce or prevent sepsis-induced immunoparalysis in a septic patient.

At least one aspect of the present technology provides one or more methods to improve respiratory function of a patient infected with a virus such as SARS-CoV-2.

At least one aspect of the present technology provides one or more systems to improve respiratory function of a patient infected with a virus such as SARS-CoV-2.

At least one aspect of the present technology provides one or more methods to reduce the duration of mechanical ventilation required for a patient infected with a virus such as SARS-CoV-2.

At least one aspect of the present technology provides one or more systems to reduce the duration of mechanical ventilation required for a patient infected with a virus such as SARS-CoV-2.

At least one aspect of the present technology provides one or more methods to reduce the duration of mechanical ventilation required for a patient suffering from sepsis.

At least one aspect of the present technology provides one or more systems to reduce the duration of mechanical ventilation required for a patient suffering from sepsis.

In certain embodiments, the outcome for a patient suffering from a viral infection, particularly an infection by SARS-CoV-2, can be improved if the patient is warmed to and/or maintained at a temperature above normal body temperature (about 37° C.) or if hyperthermia (fever) is induced mechanically.

In certain embodiments, the outcome for a patient suffering from sepsis, including bacterial sepsis, can be improved if the patient is warmed to and/or maintained at a temperature above normal body temperature (about 37° C.) or if hyperthermia (fever) is induced mechanically.

In any aspect or embodiment, the devices, methods, and systems for rapidly and efficiently managing thoracic temperature of a subject with a viral infection and/or suffering from sepsis may additionally be effective to manage core body temperature of the patient. Core body temperature can be assessed in a conventional manner, such as using a Foley catheter inserted into the patient's bladder. In some such aspects or embodiments, the devices, methods, and systems do not produce prolonged, or excessive degrees of, whole body hyperthermia, which may be detrimental to the patient.

In any aspect or embodiment, the patient is experiencing respiratory failure or at risk of experiencing respiratory failure. In some such aspects or embodiments, the patient is undergoing mechanical ventilation for the treatment of respiratory failure.

In any aspect or embodiment, the patient is undergoing mechanical ventilation.

Other features, objects, and advantages of the present invention are apparent in the detailed description that follows. It should be understood, however, that the detailed description, while indicating preferred embodiments of the invention, are given by way of illustration only, not limitation. Various changes and modifications within the scope of the invention will become apparent to those skilled in the art from the detailed description.

### BRIEF DESCRIPTION OF THE DRAWINGS

FIG. 1 shows a flowchart according to an exemplary embodiment of the present technology.

FIG. 2 shows a cross-sectional view of a heat transfer device according to an exemplary embodiment of the present technology.

FIG. 3 is a surface plot of the temperature profile for different blood perfusion rates: 1 e-3[1/s] (left panel); 3e-3[1/s] (center panel); and 6e-3[1/s] (right panel).

FIG. 4 shows a temperature profile in an xz plane at the center of the airways at different blood perfusion rates: 1 e-3[1/s] (left panel); 3e-3[1/s] (center panel); and 6e-3[1/s] (right panel).

FIG. 5 is a line graph depicting airway average and maximum temperature as a function of blood perfusion rate.

FIG. 6 is a line graph depicting lungs average and maximum temperature as a function of blood perfusion rate.

FIG. 7 depicts patient temperature across time for patients treated with an exemplary esophageal heat transfer device (targeted temperature management; TTM) and control patients.

### DETAILED DESCRIPTION OF THE INVENTION

This detailed description is intended only to acquaint others skilled in the art with the present invention, its principles, and its practical application so that others skilled in the art may adapt and apply the invention in its numerous forms, as they may be best suited to the requirements of a particular use. This description and its specific examples are intended for purposes of illustration only. This invention, therefore, is not limited to the embodiments described in this patent application, and may be variously modified.

In at least one aspect, this disclosure is directed to targeted temperature management in an individual infected with a virus, suspected of being infected with a virus, or at risk of becoming infected with a virus. Without wishing to be bound by theory, it is believed that elevated body temperature is helpful in resolving microbial and viral illnesses (e.g., by augmenting immune function, increasing production of protective heat shock proteins, directly inhibiting microorganism growth, reducing viral replication, and/or enhancing antimicrobial effectiveness); a mechanical provision of elevated temperature, particularly focused in a body region of high viral entry and activity, offers a unique therapeutic option.

In certain embodiments, targeted temperature management is effective to reduce viral load in the individual by at least 20%, at least 30%, at least 40%, at least 50%, at least 60%, at least 70%, at least 80%, or at least 90%, compared to the viral load in the individual in the absence of treatment.

In certain embodiments, targeted temperature management is employed as an adjunctive therapy. For example, the methods described herein may further comprise administering to an individual in need thereof an effective amount of anti-viral agent.

In at least one aspect, this disclosure is directed to targeted temperature management in an individual suffering from sepsis, suspected of suffering from sepsis, or at risk of developing sepsis. Without wishing to be bound by theory, it is believed that patient warming reduces immunoparalysis (by enhancing antibody production, T cell activation, and macrophage function), accelerates the heat shock response, and inhibits pathogen virulence. Therapeutic warming directly at a patient's core is hypothesized to further enhance immunomodulatory function by providing heat directly to organ systems most affected by sepsis, and via this mechanism, improve clinical outcome.

In certain embodiments, targeted temperature management is effective to reduce or prevent sepsis-induced immunoparalysis in the individual.

At least one aspect of the present technology employs a heat exchange device to manage patient temperature. Heat exchange devices can be non-invasive or invasive. For example, surface cooling can be accomplished with circulating cold water blankets and cold air-forced blankets. In certain embodiments, the heat exchange device is an esophageal heat transfer device. In certain embodiments, esophageal heat transfer devices such as those described in U.S. Pat. Nos. 8,231,664; 8,444,684; 8,523,929; 8,696,725; 9,622,909; 9,326,890; 9,301,871; 10,413,444; and 10,363,162 may be employed. The disclosures of each of the aforementioned patents are hereby incorporated by reference in their entireties.

At least one aspect of the present technology provides a method for raising a regional temperature (e.g., lung temperature and/or pulmonary vasculature temperature) in patient infected with a virus. In certain embodiments, the virus is SARS-CoV-2. The method comprises nasally or orally inserting a distal end of an esophageal heat transfer device into the patient and positioning a heat transfer region of the esophageal heat transfer device in the patient's esophagus. In certain embodiments, the heat transfer region comprises at least one lumen providing a fluid path for flow of a heat transfer medium. In some such embodiments, the method further comprises initiating flow of the heat transfer medium along the fluid path. In some such embodiments, the method further comprises circulating the heat transfer medium along the fluid path for a time sufficient to raise the regional temperature in the infected patient. In some such embodiments, the method further comprises circulating the heat transfer medium along the fluid path for at least 24 hours, at least 48 hours, or at least 72 hours. In some such embodiments, the method is also effective to raise core body temperature of the patient. In some such embodiments, the extent of the increase in core body temperature is such that regional temperature management can be maintained for a prolonged period of time, such as for at least 24 hours, at least 48 hours, or at least 72 hours.

At least one aspect of the present technology provides a method for raising a regional temperature (e.g., lung temperature and/or pulmonary vasculature temperature) in patient suffering from sepsis. In certain embodiments, the sepsis is bacterial sepsis. The method comprises nasally or orally inserting a distal end of an esophageal heat transfer device into the patient and positioning a heat transfer region of the esophageal heat transfer device in the patient's esophagus. In certain embodiments, the heat transfer region comprises at least one lumen providing a fluid path for flow of a heat transfer medium. In some such embodiments, the method further comprises initiating flow of the heat transfer medium along the fluid path. In some such embodiments, the method further comprises circulating the heat transfer medium along the fluid path for a time sufficient to raise the regional temperature in the septic patient. In some such embodiments, the method further comprises circulating the heat transfer medium along the fluid path for at least 24 hours, at least 48 hours, or at least 72 hours. In some such embodiments, the method is also effective to raise core body temperature of the patient. In some such embodiments, the extent of the increase in core body temperature is such that regional temperature management can be maintained for a prolonged period of time, such as for at least 24 hours, at least 48 hours, or at least 72 hours.

At least one aspect of the present technology provides a method for treating a patient in respiratory failure, at risk of respiratory failure, and/or mechanically ventilated. In certain embodiments, the patient is in respiratory failure, at risk of respiratory failure, and/or mechanically ventilated due to a viral infection. In certain embodiments, the patient is in respiratory failure, at risk of respiratory failure, and/or mechanically ventilated due to sepsis. The method comprises nasally or orally inserting a distal end of an esophageal heat transfer device into the patient; positioning a heat transfer region of the esophageal heat transfer device in the patient's esophagus, wherein the heat transfer region comprises at least one lumen providing a fluid path for flow of a heat transfer medium; initiating flow of the heat transfer medium along the fluid path; and circulating the heat transfer medium along the fluid path for a time sufficient to raise a regional temperature of the patient to and/or actively maintain the regional temperature of the patient at a target temperature. In certain embodiments, the regional temperature is selected from the group consisting of esophageal temperature, thoracic temperature, lung temperature, pulmonary vasculature temperature, and heart temperature. In some such embodiments, the patient is infected with a virus, such as SARS-CoV-2. In some such embodiments the patient is septic.

In certain embodiments, the esophageal heat transfer device comprises a multi-chambered silicone tube that is configured to be connected to an external source.

In certain embodiments, the esophageal heat transfer device comprises one or more lumens allowing for flow of a heat transfer medium (e.g., coolant).

In certain embodiments, the esophageal heat transfer device comprises a distal end configured for insertion into a nostril or mouth of a patient; a heat transfer medium supply tube that defines an inflow lumen and forms a heat transfer region, wherein the heat transfer region is configured to transfer heat to or extract heat from the esophagus when a heat transfer medium flows through the inflow lumen; a heat transfer medium return tube that defines an outflow lumen; an input port for receiving the heat transfer medium from a source, wherein said input port is connected to the heat transfer medium supply tube; and an output port for returning the heat transfer medium to the source, wherein said output port is connected to the heat transfer medium return tube. In some such embodiments, the heat transfer region is not inflatable. In some such embodiments, the heat transfer medium return tube is not positioned within the heat transfer medium supply tube. In some such embodiments, wherein the heat transfer medium supply tube and the heat transfer medium return tube have substantially equal inside diameters. In some such embodiments, the heat transfer medium return tube and the heat transfer medium supply tube are arranged in parallel. In certain embodiments, the esophageal heat transfer device comprises a heat transfer medium supply tube that defines an inflow lumen and a heat transfer medium return tube that defines an outflow lumen, wherein the heat transfer medium supply tube and the heat transfer medium return tube are configured such that at least a portion of an exterior wall of the heat transfer medium supply tube is in contact with esophageal tissue upon placement of the device in a patient. In some such embodiments, the portion of the exterior wall constitutes a heat transfer region configured to transfer heat to or extract heat from the esophageal tissue when a heat transfer medium flows through the inflow lumen. In some such embodiments, the heat transfer region is not inflatable. In some such embodiments, the heat transfer medium return tube is not positioned within the heat transfer medium supply tube. In some such embodiments, wherein the heat transfer medium supply tube and the heat transfer medium return tube have substantially equal inside diameters. In some such embodiments, the heat transfer medium return tube and the heat transfer medium supply tube are arranged in parallel. In some such embodiments, the heat transfer medium return tube and the heat transfer medium supply tube share a common inner wall.

In certain embodiments, the esophageal heat transfer device comprises a distal end configured for insertion into a nostril or mouth of a patient; a proximal end comprising an input port for receiving a heat transfer medium from a source and an output port for returning the heat transfer medium to the source; a length of tubing that defines a multi-lumen cavity, the length of tubing comprising an inner divider wall disposed longitudinally therein, the inner divider wall dividing the cavity into at least a first lumen and a second lumen, wherein the first lumen and the second lumen are arranged in parallel to each other, and wherein the first lumen and the second lumen are in fluid communication with each other, thereby defining a fluid path for flow of the heat transfer medium; wherein the first lumen is defined by a first circumferential portion of an interior wall of the length of tubing and the inner divider wall and the second lumen is defined by a second circumferential portion of an interior wall of the length of tubing and the inner divider wall; and wherein at least a portion of an exterior wall of the length of tubing is in contact with esophageal tissue upon placement of the device in a patient and the portion of the exterior wall constitutes a non-inflatable heat transfer region configured to transfer heat to or extract heat from the esophageal tissue when the heat transfer medium flows through the first and/or second lumen

In certain embodiments, the esophageal heat transfer device comprises a triple lumen extruded tube. In certain embodiments, the triple lumen extruded tube contains two lumens allowing for flow of a heat transfer medium (e.g., inflow and outflow). In certain embodiments, the triple lumen extruded tube contains a gastric access lumen. The gastric access lumen may, for example, allow for removal of stomach contents.

In certain embodiments, the heat transfer medium flowing along the fluid path of the esophageal heat transfer device is from about 38° C. to about 42° C. In certain embodiments, the heat transfer medium is about 38° C., about 39° C., about 40° C., about 41° C., or about 42° C.

In certain embodiments, the patient's esophageal temperature is raised to a temperature above normothermia, such as from about 38° C. to about 42° C. In certain embodiments, the patient's esophageal temperature is raised to a target temperature, such as about 38° C., about 39° C., about 40° C., about 41° C., or about 42° C. In certain embodiments, the patient's esophageal temperature is raised to a target temperature range, such as from about 38° C. to about 42° C. The proximity of the esophagus to the chief organs of circulation and respiration (e.g., heart, lungs, pulmonary vasculature) provides efficient regional temperature management of thoracic structures.

It is believed that SARS-CoV-2 is particularly susceptible to elevated temperature at least because the potential for viral entry is inhibited by elevations in temperature. The receptor binding domain (RBD) of SARS-CoV-2 has been reported to have a higher entropy penalty upon binding angiotensin-converting enzyme II (ACE2) compared to the RBD of the earlier SARS-CoV, which was first reported in February 2003. Without wishing to be bound by theory, this temperature sensitivity of SARS-CoV-2 offers a novel therapeutic approach via esophageal warming of the thorax (lungs, bronchi, pulmonary vasculature, and heart), where ACE2 receptors are located. As such, elevations in lung temperature can decrease viral replication and activity sufficiently to effect an improvement in clinical outcome.

Relevant clinical endpoints may include, but are not limited to, PaO₂/FiO₂ ratio, time on a ventilator, time to extubation, time to recovery, and mortality.

In exemplary embodiments, the disclosure provides a method for treating a patient infected with a virus, such as SARS-CoV-2. The method comprises raising a regional temperature of the patient to and/or actively maintaining a regional temperature of the patient at a target temperature. In some such embodiments, the regional temperature is the temperature of one or more thoracic structures such as the lungs and/or pulmonary vasculature. In some such embodiments, the target temperature is a range, such as from about 38° C. to about 42° C. In some such embodiments, the target temperature is maintained for a time sufficient to reduce viral load in the patient. In some such embodiments, the method further comprises establishing a diagnosis or clinical impression that the patient suffers from a viral infection. In some such embodiments, the method further comprises requesting the results of a test to detect or quantify a virus in a patient sample. In some such embodiments, the method further comprises placing a heat exchange device into an anatomical structure of the patient, such as the esophagus.

In exemplary embodiments, the disclosure provides a method for treating a patient infected with SARS-CoV-2. The method comprises raising the temperature of one or more thoracic structures of the patient to and/or actively maintaining the temperature of one or more thoracic structures of the patient at a target temperature. In some such embodiments, the thoracic structure is the lungs and/or pulmonary vasculature. In some such embodiments, the target temperature is above the normothermic range. In some such embodiments, the target temperature is above about 38.5° C. In some such embodiments, the target temperature is a range, such as from about 38° C. to about 42° C. In some such embodiments, the target temperature is maintained for a time sufficient to reduce the SARS-CoV-2 viral load in the patient. In some such embodiments, the method further comprises establishing a diagnosis or clinical impression that the patient suffers from a SARS-CoV-2 infection. In some such embodiments, the method further comprises requesting the results of a test to detect or quantify SARS-CoV-2 in a patient sample. In some such embodiments, the method comprises mechanical temperature management. In some such embodiments, the method further comprises placing a heat exchange device into an anatomical structure of the patient, such as the esophagus.

In exemplary embodiments, the disclosure provides a method for improving respiratory function in a patient infected with SARS-CoV-2 and/or reducing time on a ventilator for a patient infected with SARS-CoV-2. The method comprises raising the temperature of one or more thoracic structures of the patient to and/or actively maintaining the temperature of one or more thoracic structures of the patient at a target temperature. In some such embodiments, the thoracic structure is the lungs and/or pulmonary vasculature. In some such embodiments, the target temperature is above the normothermic range. In some such embodiments, the target temperature is above about 38.5° C. In some such embodiments, the target temperature is a range, such as from about 38° C. to about 42° C. In some such embodiments, the target temperature is maintained for a time sufficient to reduce the SARS-CoV-2 viral load in the patient. In some such embodiments, the method further comprises establishing a diagnosis or clinical impression that the patient suffers from a SARS-CoV-2 infection. In some such embodiments, the method further comprises requesting the results of a test to detect or quantify SARS-CoV-2 in a patient sample. In some such embodiments, the method comprises mechanical temperature management. In some such embodiments, the method further comprises placing a heat exchange device into an anatomical structure of the patient, such as the esophagus.

In exemplary embodiments, the disclosure provides a method for treating a patient suffering from sepsis, such as bacterial sepsis. The method comprises raising the temperature of one or more thoracic structures of the patient to and/or actively maintaining the temperature of one or more thoracic structures of the patient at a target temperature. In some such embodiments, the thoracic structure is the lungs and/or pulmonary vasculature. In some such embodiments, the target temperature is above the normothermic range. In some such embodiments, the target temperature is above about 38.5° C. In some such embodiments, the target temperature is a range, such as from about 38° C. to about 42° C. In some such embodiments, the target temperature is maintained for a time sufficient to reduce or prevent immunoparalysis in the patient. In some such embodiments, the method further comprises establishing a diagnosis or clinical impression that the patient suffers from sepsis. In some such embodiments, the method further comprises requesting the results of a test to establish a diagnosis or clinical impression that the patient suffers from sepsis. In some such embodiments, the method comprises mechanical temperature management. In some such embodiments, the method further comprises placing a heat exchange device into an anatomical structure of the patient, such as the esophagus.

In exemplary embodiments, the disclosure provides a method for improving respiratory function in a patient suffering from sepsis, such as bacterial sepsis. The method comprises raising the temperature of one or more thoracic structures of the patient to and/or actively maintaining the temperature of one or more thoracic structures of the patient at a target temperature. In some such embodiments, the thoracic structure is the lungs and/or pulmonary vasculature. In some such embodiments, the target temperature is above the normothermic range. In some such embodiments, the target temperature is above about 38.5° C. In some such embodiments, the target temperature is a range, such as from about 38° C. to about 42° C. In some such embodiments, the target temperature is maintained for a time sufficient to reduce or prevent immunoparalysis in the patient. In some such embodiments, the method further comprises establishing a diagnosis or clinical impression that the patient suffers from sepsis. In some such embodiments, the method further comprises requesting the results of a test to establish a diagnosis or clinical impression that the patient suffers from sepsis. In some such embodiments, the method comprises mechanical temperature management. In some such embodiments, the method further comprises placing a heat exchange device into an anatomical structure of the patient, such as the esophagus.

Certain example embodiments of the presently described technology now will be described with respect to the appended figures; however, the scope of the present technology is not intended to be limited thereby. It is to be understood that the scope of the present technology is not to be limited to the specific embodiments described herein. The technology may be practiced other than as particularly described and still be within the scope of the claims.

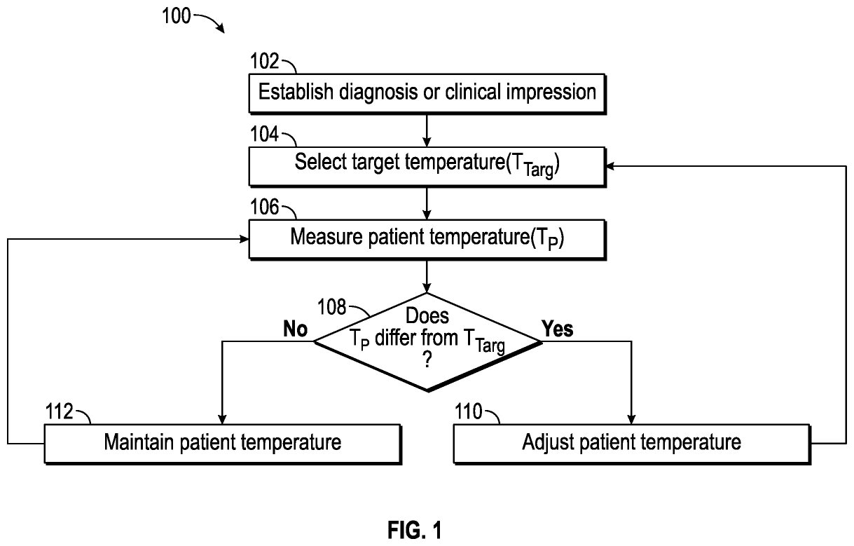

FIG. 1 shows a flow diagram of a method 100 for the use of targeted temperature management to treat viral infection, reduce viral load, and/or treat sepsis, such as bacterial sepsis. According to various embodiments, at least a portion of the activity described with respect to FIG. 1 may be implemented via one or more heat exchange devices described herein.

As shown at 102, an initial diagnosis or clinical impression is established (e.g., viral infection, sepsis). Such diagnosis or clinical impression may be reached on the basis of a physical examination, a patient history, and/or one or more laboratory tests. In certain embodiments, a diagnosis or clinical impression that the patient is infected with a virus is reached in the absence of a laboratory test. In certain embodiments, a diagnosis or clinical impression that the patient is infected with a virus is based, at least in part, on the results of a laboratory test. In certain embodiments, a healthcare provider requests the results of a laboratory test to detect or quantify a virus in a patient sample. In certain embodiments, the laboratory test is an immunoassay, such as an enzyme-linked immunosorbent assay (ELISA) or Western blot. In certain embodiments, the laboratory test detects and/or quantifies a viral antigen. For example, a viral antigen may be detected and/or quantified by ELISA. In certain embodiments, the laboratory test detects and/or quantifies a virus-specific immunoglobin, such as immunoglobin M (IgM) or G (IgG). For example, a virus-specific IgM or IgG may be detected and/or quantified by ELISA or Western blot. In certain embodiments, the laboratory test detects and/or quantifies a viral genome or portion thereof. For example, a viral genome or portion thereof may be detected and/or quantified by reverse transcription-polymerase chain reaction (RT-PCR). In certain embodiments, the laboratory test is a viral load test. For example, viral load may be determined by PCR, branched-chain DNA, or nucleic acid sequence based amplification (NASBA). In certain embodiments, the patient sample is a blood sample, such as a serum sample. In certain embodiments, the patient sample is a saliva sample.

After it has been determined that the patient suffers from a viral infection and/or sepsis, a target temperature (T_{Targ}) is selected, as indicated by block 104. In certain embodiments, a temperature management system enables a user to select a target temperature (T_{Targ}). In certain embodiments, the target temperature is a threshold value or a range. In certain embodiments, the target temperature includes an upper and/or a lower limit. In certain embodiments, the target temperature is between about 37° C. and about 42° C. In certain embodiments, the target temperature is greater than or equal to 38.5° C., alternatively greater than or equal to 39.5° C. In certain embodiments, the target temperature includes a normothermic range.

Optionally, patient temperature (T_{P}), such as esophageal temperature, is measured, as indicated by block 106. In certain embodiments, a temperature management system enables a user to determine T_{P}.

As indicated by decision diamond 108, T_{P} is compared to T_{Targ}. In this way, patient temperature may be managed irrespective of the patient's initial temperature. In certain embodiments, a temperature management system enables a comparison between T_{P} and T_{Targ}. If T_{P} differs from T_{Targ}, a heat exchange device is employed to adjust the patient's temperature, as shown in block 110. If T_{P} does not differ from T_{Targ}, a heat exchange device is employed to maintain the patient's temperature, as shown in block 112. In certain embodiments, the patient's temperature is maintained at (or within) the target temperature for a period of time. In certain embodiments, the period of time is from about eight (8) hours to about seventy-two (72) hours. In certain embodiments, the period of time is about forty-eight (48) hours. In certain embodiments, the period of time is at least forty-eight (48) hours. In certain embodiments, the period of time is at least seventy-two (72) hours

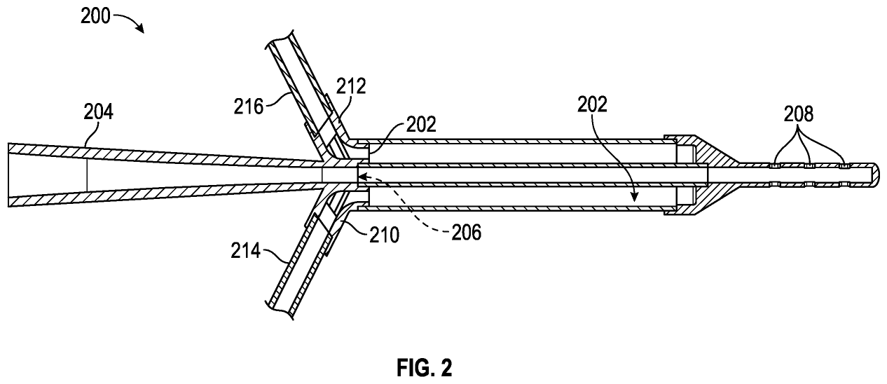

FIG. 2 is a cross-sectional view of an exemplary heat transfer device 200. The heat transfer device 200 comprises an internal cavity 202 and a gastric access tube 204. The gastric access tube 204 defines a gastric access lumen 206. The distal section of the heat transfer device 200 includes one or more ports 208 along the side of the gastric tube 204. The one or more ports 208 may provide for communication between the space exterior to the device 200 and the gastric access lumen 206. For example, the one or more ports 208 may act as a portal between the patient's stomach and the gastric access lumen 206 allowing the gastric contents to be suctioned from the patient's stomach out through the gastric access lumen 206. The presence of multiple ports 208 provides reduced likelihood of blockage of the gastric access lumen 206 from semi-solid stomach contents. Alternatively, multiple gastric access lumens may be employed. The addition of one or more ports 208 may improve and enhance the removal of stomach contents, which, in turn, may improve contact between gastric mucosa and the heat transfer device 200. Such improved contact may enhance heat transfer between the heat transfer device 200 and the gastric mucosa. The configuration of the ports 208 shown in FIG. 2 is oval. However, the ports 208 can be, for example, circular, rectangular, or any other shape that permits flow of gastric contents from the stomach to the gastric access lumen 206.

The heat transfer device 100 includes an input port 210 and an output port 212. Input port 210 may be connected to an inflow tube 214. Inflow tube 214 may, for example, carry heat transfer medium from an external source (not shown) to input port 210. Output port 212 may be connected to an outflow tube 216. Outflow tube 216 may, for example, carry heat transfer medium from device 200 to an external source (not shown).

Prior to undertaking the studies described herein, it was not clear whether an esophageal heat transfer device could be employed in a mode sufficient to achieve patient benefit vis-à-vis regional (e.g., thoracic) temperature management without adversely impacting the patient in other ways, such as prolonged, or excessive degrees of, whole body hyperthermia.

### EXAMPLE 1

### Mathematical Modeling

As an initial investigation into the approach described herein, a mathematical model of core warming was developed to determine the temperature distribution throughout lung tissue at typical physiologic conditions.

The software COMSOL Multiphysics® was used to model and simulate heat transfer in the body from an intraesophageal warming device, taking into account the airflow from patient ventilation. The simulation was focused on heat transfer and warming of the lungs and performed on a simplified geometry of an adult human body and airway from the pharynx to the lungs. This symmetry was utilized to reduce the computational cost, so only half of the body was simulated.

The Bioheat Transfer interface of COMSOL Multiphysics® was used for the modeling and simulation of heat transfer, considering the heat generated by the blood perfusion, and known tissue thermal properties. The bioheat transfer governing equations are given by Equation 1, where T is the temperature and is the dependent variable, p is the density, Cp the heat capacity, k the thermal conductivity, μ the velocity field (which is obtained from the fluid flow in the airways) and Qbio is the heat generation term with “b” being a subscript corresponding to blood. As the intraesophageal device temperature is higher than the blood temperature in this simulation (Tb=37° C.), this term has a negative sign, making it a heat consuming term. The temperature boundary condition at the warming device inlet was set to 42° C., the maximum operating temperature of the device. The surrounding air at 20° C. (emulating a hospital environment) was not considered in the model, but replaced with a convection boundary condition at the skin boundaries.

$$\underbrace{ρ {C}_{p} \frac{\partial T}{\partial t}}_{Transient} + \underbrace{ρ {C}_{p} u \cdot ∇ T}_{Convection} + \underbrace{∇ \cdot ( - k ∇ T )}_{\begin{matrix}Diffusion or \\ Conduction\end{matrix}} = \underbrace{{Q}_{bio} = {ρ}_{b} {C}_{p , b} {ω}_{b} ( {T}_{b} - T )}_{Blood Perfusion Heat Generation} \qquad Equation 1$$

The airflow was modeled using the Turbulent Flow, k-c interface considering a stationary study with the RMS value of 0.5[m/s] of a presumed sinusoidal behavior of the airflow velocity at the inlet of the trachea at the throat level. The boundary condition in the airflow outlets was set to pressure of −1 [mmHg]. The warming water flow was directly defined to be 120 L/h in the negative z-axis direction, which corresponds to the actual flow rate in the clinical use of the device.

Blood perfusion rate sensitivity analysis: The simulations were run considering a range of values for the blood perfusion rate, which is a parameter expected to have high influence in the heat consumption term in Equation 1, since the heat capacity and density remains almost constant. The values considered for the blood perfusion rate were 1 e-3[1/s] to 6e-3[1/s] with a step of 1 e-3[1/s].

The simulation results show a temperature distribution which agrees with the expected clinical experience, with the skin surface at a lower temperature than the rest of the body due to convective cooling in a typical hospital environment; however, most of the torso is at 37° C. in the healthy-condition body temperature. The highest temperature in this case is the device warming water temperature, and that heat diffuses by conduction to the nearby tissues, including the air flowing in the airways. With the effective (RMS) value considered for the airflow, warmed air moves through the lungs, and the intraesophageal warming device leads to an increment in the core temperature, as well as in the bronchi from the trachea to the lungs.

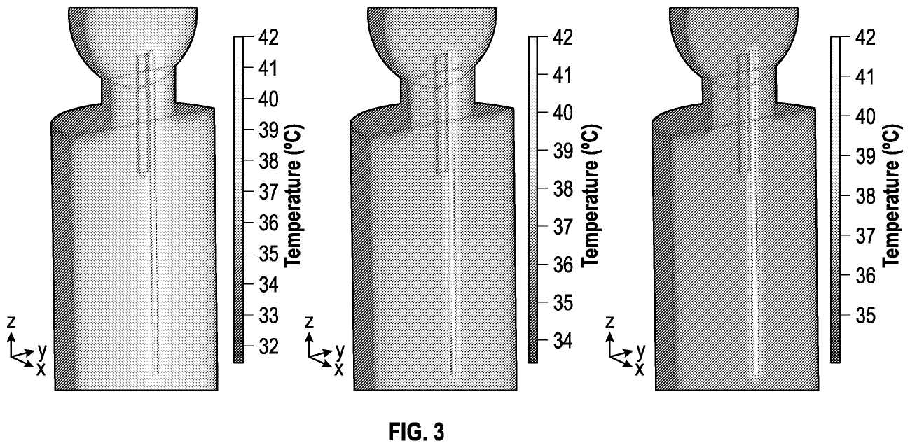

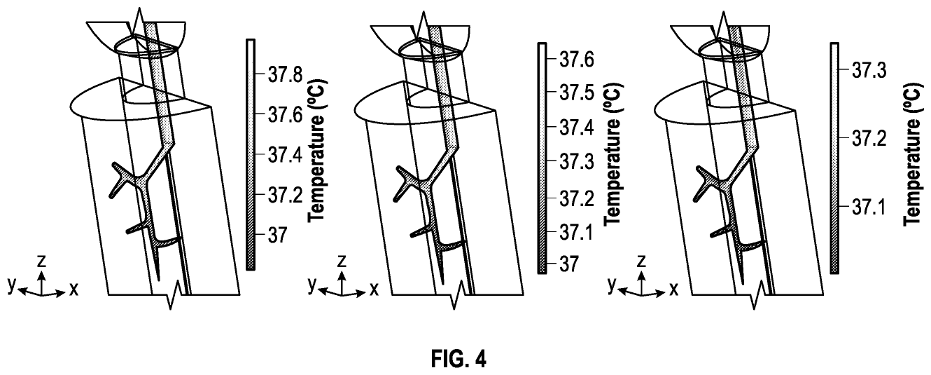

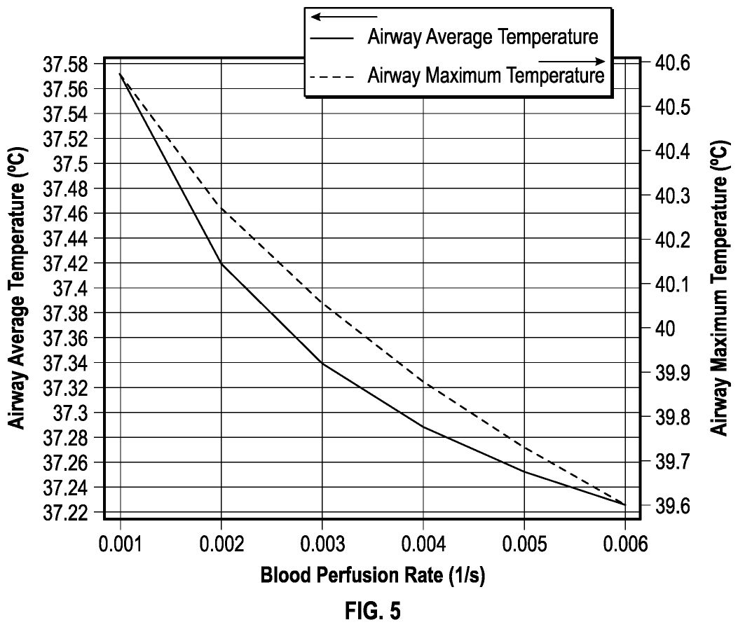

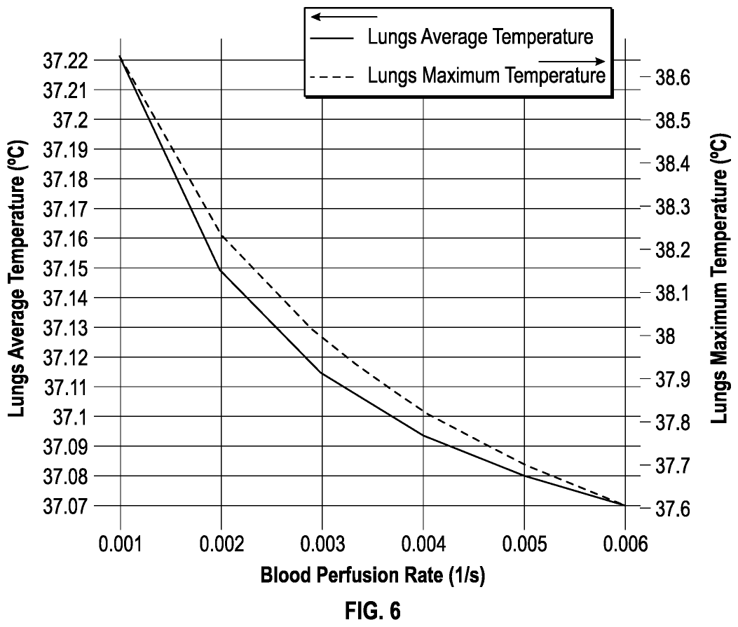

The airflow velocity profile is as expected, being around 0.5[m/s] in the trachea and getting higher as the diameter of the airway decreases. From FIG. 3, it is evident that the blood perfusion rate affects the temperature distribution, with the heat transfer from the warming device increasing, and the skin temperature decreasing, with a reduced blood perfusion rate. This is seen in FIG. 4, where heat diffuses to the air in the trachea more rapidly, and the temperature has higher values, at lower blood perfusion rates. The relationship between both the maximum and average temperatures with respect to the blood perfusion rate is exponential in the airways (FIG. 5) as well as in the lungs (FIG. 6). These results are expected given that the blood perfusion heat generation term is a heat consumption term in the warming case considered (Equation 1). At the expected ranges of perfusion in the patient requiring critical-care, the temperature of lung tissue, including vasculature and airways, can be elevated to a range sufficient to offer therapeutic benefit.

Conclusions. The provision of core warming via commercially available technology commonly utilized in the intensive care unit, emergency department, and operating room can increase regional temperature of lung tissue and airway passages. The characteristics of many viruses, and in particular, the temperature sensitivities of SARS-CoV-2, combined with increasing evidence of potential benefits of elevated body temperature in treating infectious critically-ill patients requiring mechanical ventilation suggests that the use of focused, core warming may offer a novel means of improving outcome of COVID-19 that warrants further study.

### EXAMPLE 2

### Core Warming of COVID-19 Patients Undergoing Mechanical Ventilation: A Randomized, Single Center Pilot Study

The purpose of the study was to determine if core warming improves respiratory physiology of mechanically ventilated patients with COVID-19, allowing earlier weaning from ventilation, and greater overall survival. Patients with a diagnosis of COVID-19 on mechanical ventilation and a maximum baseline temperature (within previous 12 hours) <38.3° C. were included in the study.

This was a small scale pilot study to evaluate if core warming via an esophageal heat transfer device improved respiratory physiology of mechanically ventilated patients with COVID-19, allowing earlier weaning from ventilation, and greater overall survival. This prospective, randomized study included 20 patients diagnosed with COVID-19, and undergoing mechanical ventilation for the treatment of respiratory failure.

The esophageal heat transfer device was a single use thermal regulating device that is placed in the esophagus and connected to an external heat exchange unit, creating a closed-loop system for heat transfer to increase or decrease patient temperature. Its placement in the esophagus, with proximity to blood flow from the heart, pulmonary vasculature, and great vessels, allowed efficient heat transfer while an open central lumen allows gastric decompression, medication administration, or tube feeding.

Patients were randomized in a 1:1 fashion with 10 patients (Group A) randomized to undergo core warming, and the other 10 patients (Group B) serving as the control group who did not have the esophageal heat transfer device used. Patients randomized to Group A had core warming initiated in the ICU or other clinical environment in which they were being treated.

The esophageal heat transfer device was set to 42° C. after initial placement, and maintained at between 38° C. and 42° C. for the duration of treatment. For the majority of patients, the esophageal heat transfer device was maintained at 42° C. for the duration of treatment.

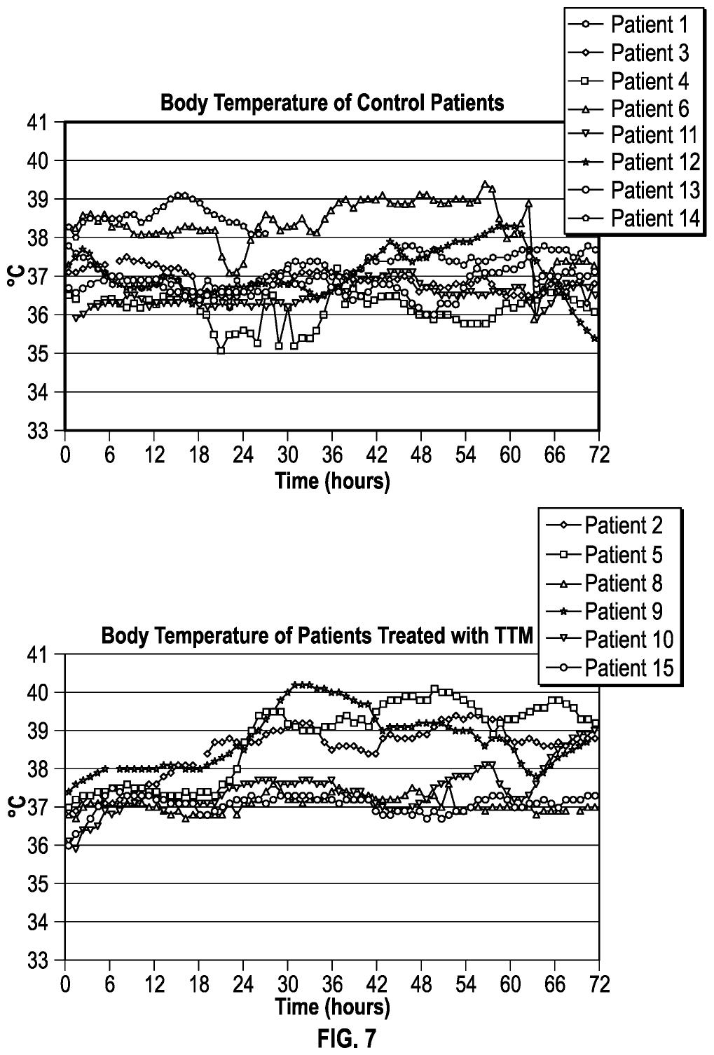

FIG. 7 shows patient core body temperature, as measured by a Foley catheter placed in the patient's bladder, for the first 14 patients enrolled in the study (patient 7 was a screen failure). Unexpectedly, the average patient core body temperature (as measured by a Foley catheter) did not rise above 39° C. over 72 hours, indicating that regional warming via the esophageal route could safely be achieved without producing prolonged, or excessive degrees of, whole body hyperthermia, which may be detrimental to the patient. Indeed, the maximum core body temperature achieved by any patient during the treatment period was 40.2° C. That patient's core body temperature was above 40° C. for about 7 hours. One other patient's core body temperature briefly exceed 40° C. None of the other remaining patients in the warming group experienced core body temperatures above 40° C. for the duration of the treatment period (72 hours). In fact, no other patient registered a core body temperature above 39.5° C. for the duration of the treatment period.

Additional outcomes to be assessed include PaO₂/FiO₂ ratio, which is measured at initiation of core warming and 24, 48, and 72 hours after initiation of core warming; viral load, which is measured 72 hours after initiation of core warming; duration of mechanical ventilation; and mortality.

### EXAMPLE 3

### Working Toward the Advancement of Recovery Using Modulated Therapeutic Hyperthermia in Sepsis

The purpose of the study is to determining the effect size and the dose-response relationship between patient temperature (controlled by therapeutic core warming) and clinical outcome to identify optimal therapeutic treatment targets and uncover possible threshold effects.

This is a response-adaptive randomized, controlled trial. Patients with a diagnosis of sepsis where mechanical ventilation is expected for more than 24-72 hours and a maximum baseline temperature (within previous 12 hours) <38.3° C. are included in the study.

Afebrile patients suffering from sepsis and requiring mechanical ventilation are enrolled in a 3-arm adaptive design. Control arm patients receive standard care, while treatment arm patients undergo 72 hours of core warming to maximum temperatures of either 38.5° C. or 39.5° C. Patients are assigned an arm corresponding to either of two warming groups (Groups A: 38.5° C. target, or Group B: 39.5° C. target) or standard care (Group C: Control). A maximum patient temperature of 40° C. is enforced by reducing device temperature as needed, and a dose-response is measured.

The esophageal heat transfer device is a single use thermal regulating device that is placed in the esophagus and connected to an external heat exchange unit, creating a closed-loop system for heat transfer to increase or decrease patient temperature. Its placement in the esophagus, with proximity to blood flow from the heart, pulmonary vasculature, and great vessels, allows efficient heat transfer while an open central lumen allows gastric decompression, medication administration, or tube feeding.

Insertion of the esophageal heat transfer device is in place of the standard orogastric tube utilized in mechanically ventilated patients. Placement occurs within 4 hours of enrollment. The device is used as indicated (for warming), and patient body temperature measurements are collected using standard thermometers (Foley, rectal, etc.). The device is set to 42° C. temperature after initial placement, and maintained at 42° C. for the duration of treatment or until maximum patient target temperature is reached. Patient temperature in the treatment arms is allowed to increase from baseline to either 38.5° C. (Group A) or to 39.5° C. (Group B). If patient temperature increases above this range, the device is set to an operating temperature of 38.5° C. or 39.5° C., respectively, thereby preventing any further increase in patient temperature. In the event of further patient temperature increase despite reduced warming (signaling innate fever response), the esophageal heat transfer device is turned off, but antipyretic treatment is reserved for patients exceeding 40° C. All patients are to have usual standard of care labs, vital signs, and imaging obtained from patients undergoing mechanical ventilation in the ICU. Baseline demographics, comorbid conditions, hemodynamic parameters, hourly body temperature measurements, and Sequential Organ Failure Assessment (SOFA) scores are recorded. Specific additional parameters to be measured include PaO₂ at regular intervals and FiO₂ at the time of obtaining blood gases for PaO₂ measurement, to allow calculation of P/F ratio.

Organ support-free days (days alive and free of ICU-based respiratory or cardiovascular support within 21 days, where patients who die are assigned −1 day) are assessed.

The intervention will result in a statistically and clinically significant improvement in patient outcome as measured by organ support-free days with core warming, with greater warming showing greater benefit. Demonstration of this outcome supports use of the esophageal heat transfer device for both the treatment of sepsis and for further clinical investigation around the influence of active therapeutic warming of patients with sepsis.

Development of nosocomial infections and impact on sepsis-induced immunoparalysis as measured by absolute lymphocyte counts and TNF-alpha/IL-10 ratio also is assessed as described below.

### EXAMPLE 3.1

### Impact of Core Warming on the Development of Nosocomial Infections

The rationale for this assessment is to determine the relationship between nosocomial infections and body temperature.

Each patient enrolled in the study is monitored for nosocomial infections at four sites (microbiologically documented pulmonary, urinary tract, bloodstream, and catheter-related infections). Specific infections of interest are those comprising the categorization by the Centers for Disease Control and Prevention: Central line-associated bloodstream infections (CLASSI), Catheter-associated urinary tract infections (CAUTI), Surgical site infections (SSI), and Ventilator-associated pneumonia (VAP).

The intervention will result in a reduction in the development of nosocomial infections, which provides mechanistic understanding of the effects of temperature on patient outcome.

### EXAMPLE 3.2

### Impact of Core Warming on Sepsis-Induced Immunoparalysis

The rationale for this assessment is to understand the impact of therapeutic warming on the immune status of patients.

Sepsis-induced immunoparalysis is assessed and compared between the two groups by measuring absolute lymphocyte counts and IL-10 to TNF-α ratios at regular intervals over the intervention period (at baseline, and then at 24, 48, and 72 hours after treatment initiation). Immunological testing is performed by a blinded research laboratory technician.

The intervention will result in a dose-response effect between patient temperature and measures of immunoparalysis, which provides further mechanistic understanding of the effects of temperature on patient outcome.

It will be readily apparent to those skilled in the art that other suitable modifications and adaptations of the compositions and methods of the invention described herein may be made using suitable equivalents without departing from the scope of the invention or the embodiments disclosed herein.

As used herein, the words “a,” “an,” and “the” mean “one or more,” unless otherwise specified. In addition, where aspects of the present technology are described with reference to lists of alternatives, the technology includes any individual member or subgroup of the list of alternatives and any combinations of one or more thereof.

The disclosures of all patents and publications, including published patent applications, are hereby incorporated by reference in their entireties to the same extent as if each patent and publication were specifically and individually incorporated by reference.

It is to be understood that the scope of the present invention is not to be limited to the specific embodiments described above. The present invention may be practiced other than as particularly described and still be within the scope of the accompanying claims.

1. A method for treating a patient infected with a virus, the method comprising raising a regional temperature of the patient to and/or actively maintaining a regional temperature of the patient at a target temperature.

2. The method of claim 1, wherein the regional temperature is selected from the group consisting of esophageal temperature, thoracic temperature, lung temperature, pulmonary vasculature temperature, and heart temperature.

3. The method of claim 1, wherein the target temperature is above the normothermic range.

4. The method of claim 1, wherein the target temperature is a range, preferably from about 38° C. to about 42° C.

5. The method of claim 1, wherein the target temperature is maintained for a time sufficient to reduce viral load in the patient.

6. The method of claim 1, further comprising requesting the results of a test to detect or quantify a virus in a patient sample and/or establishing a diagnosis or clinical impression that the patient suffers from a viral infection.

7. The method of claim 1, further comprising placing a heat exchange device into an anatomical structure of the patient.

8. The method of claim 7, wherein the anatomical structure is an esophagus and the heat exchange device is an esophageal heat transfer device.

9. The method of claim 1, wherein the virus is SARS-CoV-2.

10. The method of claim 1, wherein the patient is in respiratory failure, at risk of respiratory failure, and/or mechanically ventilated.

11. A method for treating a patient suffering from sepsis, the method comprising nasally or orally inserting a distal end of an esophageal heat transfer device into the patient; positioning a heat transfer region of the esophageal heat transfer device in the patient's esophagus, wherein the heat transfer region comprises at least one lumen providing a fluid path for flow of a heat transfer medium; initiating flow of the heat transfer medium along the fluid path; and circulating the heat transfer medium along the fluid path for a time sufficient to raise a regional temperature of the septic patient to and/or actively maintain the regional temperature of the septic patient at a target temperature.

12. The method of claim 11, wherein the regional temperature is selected from the group consisting of esophageal temperature, thoracic temperature, lung temperature, pulmonary vasculature temperature, and heart temperature.

13. The method of claim 11, wherein the target temperature is above the normothermic range.

14. The method of claim 11, wherein the target temperature is a range, preferably from about 38° C. to about 42° C.

15. The method of claim 11, wherein the patient is suffering from bacterial sepsis.

16. The method of claim 11, wherein the patient is in respiratory failure, at risk of respiratory failure, and/or mechanically ventilated.

17. A method for treating a patient in respiratory failure, at risk of respiratory failure, and/or mechanically ventilated due to a viral infection and/or sepsis, the method comprising nasally or orally inserting a distal end of an esophageal heat transfer device into the patient; positioning a heat transfer region of the esophageal heat transfer device in the patient's esophagus, wherein the heat transfer region comprises at least one lumen providing a fluid path for flow of a heat transfer medium; initiating flow of the heat transfer medium along the fluid path; and circulating the heat transfer medium along the fluid path for a time sufficient to raise a regional temperature of the patient to and/or actively maintain the regional temperature of the patient at a target temperature, wherein the regional temperature is selected from the group consisting of esophageal temperature, thoracic temperature, lung temperature, pulmonary vasculature temperature, and heart temperature.

18. The method of claim 17, wherein the patient is infected with a virus.

19. The method of claim 18, wherein the virus is SARS-CoV-2.

20. The method of claim 17, wherein the patient is septic.
